(19) United States
(12) Patent Application Publication
(10) Pub. No.: US 2002/0136691 A1
(43) Pub. Date: Sep. 26, 2002

(54) Pharmacological evaluation test (PET) in animals for continuous assessment of physiological functions

(76) Inventors: Paul C. Harrison, Naugatuck, CT (US); Hans J. Schierok, Warthausen (DE)

Correspondence Address: BOEHRINGER INGELHEIM CORPORATION, 900 RIDGEBURY ROAD, P O BOX 368, RIDGEFIELD, CT, 06877, US

(21) Appl. No.: 09/945,899

(22) Filed: Sep. 4, 2001

Related U.S. Application Data
(60) Provisional application No. 60/229,692, filed on Sep. 1, 2000.

(51) Int. Cl. A61K 49/00; G06F 19/00; G01N 33/48; G01N 33/50

(52) U.S. Cl. 424/9.2; 702/19

(57) ABSTRACT

The present invention provides a pharmacological evaluation test (PET) model for evaluating candidate compounds for physiological and pharmacological effects in a single animal. Test systems and pharmacological functions which can be assessed using the present invention include but are not limited to: central nervous system (e.g., behavioral, neurologic and autonomic overt effects using, for example, a modified Irwin profile); cardiovascular and central nervous systems (e.g., blood pressure, heart rate, motility using, for example, a telemetric system); pulmonary system (e.g., frequency and tidal volume using, for example, a chamber plethysmograph technique); renal function (e.g., urine output, creatinine excretion, and electrolyte excretion using, for example, metabolic cages); complete blood cell and white blood cell differential counts; clinical chemistries indicative of liver and kidney function; blood plasma and brain tissue for compound level determinations; organ weight ratios; metabonomics; and proteomics; genomics and imaging evaluations.

### RELATED APPLICATION

The benefit of prior provisional application Ser. No. 60/229,692, filed Sep. 1, 2000 is hereby claimed.

### BACKGROUND

The present invention provides a pharmacological evaluation test (PET) model for evaluating candidate compounds for physiological and pharmacological effects in a single animal. The present system provides for rapid, simultaneous generation of a wide range of relevant physiological parameters in conscious animals with a modest requirement for candidate compound.

Previous methods have relied on subjective observation (e.g., Irwin profiling or Functional Observation Battery (FOB)) (Irwin, S. 1968, Psychopharmacologia 13:222-257; Moser, V C. 1989, J. Am. Coll. Toxicol. 8:85-93); measurement of a single effect per animal (i.e., separate tests for each organ system) (Chong et al., 1998, J. Pharm. Tox. Methods 163-168; Deveney et al., 1998, J. Pharm. Tox. Methods 71-79); and measurement in restrained animals (Kissinger et al., EP 0 986 951 A2, filed September 17, 1999). Thus, a need exists for a method to obtain physiological and pharmacological information from the same subject, where each measure exists in quantitative relationship to the other and where the conditions for measurement are the same.

### SUMMARY OF THE INVENTION

The present invention provides a pharmacological evaluation test (PET) model for evaluating candidate compounds for physiological and pharmacological effects in a single animal. Test systems and pharmacological functions which can be assessed using the present invention include but are not limited to: central nervous system (e.g., behavioral, neurologic and autonomic overt effects using, for example, a modified Irwin profile); cardiovascular and central nervous systems (e.g., blood pressure, heart rate and motility using, for example, Data Sciences International (DSI) telemetric system); pulmonary system (e.g., frequency and tidal volume using, for example, a BUXCO chamber plethysmograph technique); renal function (e.g., urine output, creatinine excretion, and electrolyte excretion using, for example, metabolic cages). Additional parameters which can be assessed include: complete blood cell and white blood cell differential counts; clinical chemistries indicative of liver and kidney function; blood plasma and brain tissue for compound level determinations; organ weight ratios; metabonomics; proteomics; and genomics. Finally, examination of intact tissues using various imaging techniques (e.g., magic angle spinning NMR, PET scanning, MRI, or bone scanning using histomorphometric analysis) can be assessed in a snapshot manner at the end of a study.

### BRIEF DESCRIPTION OF THE DRAWINGS

FIG. 1. Mean frequency results from an acute (single dose) PET experiment. Male Sprague Dawley rats were administered saline (control) (5 ml/kg, p.o., -▪-), d-amphetamine (2mg/kg, p.o., -●-) or d-amphetamine (8 mg/kg, p.o, -▴-). Respiratory frequency was measured using a BUXCO chamber.

FIG. 2. Mean tidal volume results from an acute (single dose) PET experiment. Male Sprague Dawley rats were administered saline (control) (5 ml/kg, p.o., -▪-), d-amphetamine (2mg/kg, p.o., -●-) or d-amphetamine (8 mg/kg, p.o, -▴-). Tidal volume was calculated from recordings using a BUXCO chamber.

FIG. 3. Effects of prednisolone on normal body weight gain from a subacute (5 doses; 1 dose/day) PET experiment. Male Sprague Dawley rats were administered CMC/Tween (control) (5 ml/kg, p.o.; -▪-, -∘- and -▴-), prednisolone (300 mg/kg, p.o.; -□-, -∘- and -Δ-) or prednisolone (1000 mg/kg, p.o.; -+- and -×-). P-0, P-1, etc. represents days post-surgery; D-1, D-2, etc. represents days of prednisolone dosing. Individual rat body weights were recorded daily in grams using an Ohaus Navigator™ digital balance.

FIG. 4. Urine flow results from an acute (single dose) PET experiment. Male and female Sprague Dawley rats were administered 0.5% methocel (control) (5 ml/kg, p.o., solid black bars), furosemide (10 mg/kg, p.o., diagonal lined bars) or furosemide (30 mg/kg, p.o., cross hatched bars). Urine flow rate (μL/minute 100g) was calculated from the weight of the volume of urine collected using metabolism cages over the time intervals indicated.

FIG. 5. Blood pressure effects from an acute (single dose) cardiovascular experiment. Male Wistar rats were administered 1% natrosol 250 (control)(5 ml/kg, p.o., -∘-), nifedipine (1 mg/kg, p.o., -▪-), nifedipine (3 mg/kg, p.o., -567-) or nifedipine (10 mg/kg, p.o., -568 -). Blood pressure was measured telemetrically using DSI transmitters.

### DETAILED DESCRIPTION OF THE INVENTION

The Pharmacological Evaluation Test (PET) methods of the present invention comprise a series of tests designed to provide a pharmacological safety profile of compounds using the same group of animals. This pharmacological safety profile concentrates on the physiological (functional/quantitative) readouts from a number of organ systems which can more than satisfy the “core battery” of tests specified in the ICHS7A guidelines (International Conference on Harmonization of Technical Requirements for Registration of Pharmaceuticals for Human Use; Safety Pharmacology Studies for Human Pharmaceuticals). Organ systems which can be assessed include: central nervous system (e.g., behavioral, neurological and autonomic overt effects using, for example, a modified Irwin procedure, and motility measurements using, for example, a telemetry recording system), cardiovascular system (e.g., blood pressure, heart rate and ECG using, for example, a telemetry recording system), pulmonary system (e.g., frequency, tidal volume, minute volume, inspiratory time, expiratory time, peak inspiratory flow, peak expiratory flow end inspiratory pause, end expiratory pause, enhanced pause, accumulated volume and other derived parameters using, for example, either whole body plethysmograph chambers, or head out plethysmograph chambers) and renal system (urine output and creatinine excretion and electrolyte [Na⁺, K⁺ and Cl⁻] excretions using, for example, metabolic cages). In one embodiment of the invention, cardiovascular and motility parameters can be recorded continuously, the renal function can be assessed over various blocks of time (e.g., 0-4, 4-8, 8-24 hours, etc.). The pulmonary system can be assessed over a block of time (e.g., 1, 2, 4 hours, etc.) bracketing the Tmax (time to highest plasma concentration of an administered compound) or Cmax (maximal concentration of an administered compound in plasma). Central nervous system evaluations (e.g., modfied Irwin) can be performed in a snapshot fashion at the Tmax, or can be performed several times throughout the study period. Other parameters which can be assessed in snapshot fashion in the same animals during a study may include: blood collection for compound plasma level determinations, complete blood counts (CBC) and differential counts for assessment of hematological effects and serum clinical chemistries for indications of effects on the liver and kidneys; organ weight ratio determinations (e.g., liver and spleen to body weight and/or brain weight) for effects on these organs; brain collection for compound brain level determinations and tissue and/or fluid collections for use in histology, metabonomic, proteomic, genomic and/or imaging evaluations.

In addition to satisfying the “core battery” of tests specified in the ICHS7A, the Pharmacological Evaluation Test as a single test or study can fulfill the requirement that the “core battery” studies be conducted under Good Laboratory Practice (GLP) more easily than a number of separate studies. Safety pharmacology “core battery” tests, which include studies on the central nervous system, cardiovascular system and pulmonary system, are generally performed in separate studies requiring separate protocols, study monitors, compound preparations, etc. With the PET system of the present invention, GLP requirements need only to be monitored and satisfied once.

Another benefit resultant from performing the “core battery” of tests in a single PET study is that fewer animals are required (and as a consequence less compound is necessary) in comparison to performing multiple studies independently. Use of fewer animals to obtain the same or equivalent information is encouraged by the United States Department of Agriculture (USDA) in their Animal Care Policies booklet dated Apr. 14, 1997, policy #12 where they encourage a policy of “reduction, replacement and refinement (the three R's).” These USDA policies reflect current implementation of the Animal Welfare Act (9 Code of Federal Regulations Part II Department of Agriculture).

Another benefit resultant from the system of the present invention is the use of conscious animals in contrast to anesthetized animals thereby eliminating an unnecessary strain to the animal.

Applications of the PET system may include, but are not limited to: a “standard” protocol where both acute (single dose) and subacute (5 doses; 1 dose/day) dosing regimens are evaluated in all parts of the test system, a “truncated” protocol where only the acute dosing regimen is studied for compounds that may have already been evaluated, for example, in multiple dosing toxicology studies, and a “screening” protocol where only the acute or the subacute dosing regimen is evaluated, for example, with compounds early in the discovery phase.

Animals

Animals that can be used for the PET system of the invention include, but are not limited to: mice, rats, guinea pigs, rabbits, dogs, non-human primates, and sheep. Animals of both sexes can be studied using the PET system.

Administration of Compounds

Compounds can be administered by any suitable route including, but not limited to: oral, intraperitoneal, intradermal, intramuscular, subcutaneous, intranasal, and intravenous. The compounds may be administered together with other biologically active agents and/or with a pharmaceutically acceptable carrier. Such a carrier includes but is not limited to saline, buffered saline, dextrose, water, glycerol, ethanol, and combinations thereof.

Standard Protocol

For a “standard” protocol, half of the animals to be used are placed in the acute pulmonary function/CNS evaluation arm of the study and the other half of the animals are placed in the subacute cardiovascular/renal arm of the study. Animals in both arms can be further divided into subgroups, which are dosed with vehicle or compound at various doses. Animals in the pulmonary function/CNS evaluation arm of the study can be administered a single dose of vehicle or compound. After dosing, a battery of pharmacologic tests including pulmonary function assessment and CNS assessment are performed. Animals in the cardiovascular/renal function arm of the study having telemetry implants are administered daily doses of vehicle or compound for several days, e.g. for 5 days. Blood pressure, heart rate and motility can be continuously monitored via telemetry recording, for example, 24 hours/day over 4 days. Urine can be collected post-dosing and assessed for changes in volume output, electrolyte excretion, and metabonomic results (e.g. nuclear magnetic resonance spectra). In one embodiment, urine is collected 0-8 and 8-24 hours post dosing on days 1 and 4. Pulmonary function/CNS evaluations can be performed on these animals as previously described, for example, on day 5. At appropriate times, blood and/or tissues can be collected.

Cardiovascular and Motility Monitoring

Surgery:

For the cardiovascular/renal function arm, a transmitter for monitoring pressure can be placed in a blood vessel or heart chamber of each animal. Transmitters which may be used include, inter alia, a Data Sciences International (DSI, St Paul, Minn.) TA11PA-C40 implant. Alternatively, a TL11M2-C50-PXT implant could be used if continuous ECG or body temperature monitoring is required. Depending on the species of animal and type of experiment, transmitters can be placed in a heart chamber or blood vessel, including, but not limited to: the abdominal aorta, femoral artery, carotid artery, or aorta. Cardiovascular function data and motility parameters can be recorded in conscious freely moving animals using, for example, DSI telemetry recording equipment and analyzed using, for example, SAS software. On the day of recording, the animals are placed into metabolism cages for simultaneous urine collection. Parameters that can be monitored include blood pressure (BP), heart rate (HR) and motility. Measurements are taken at scheduled intervals (e.g., 10 sec. of data collection once every 2 minutes for each animal over 24 hours). An appropriate baseline period of at least 30 minutes is collected prior to animal dosing. For dosing, animals are briefly removed from their cage, weighed, dosed and returned.

Blood pressure, heart rate and activity can be measured at specified time-points, for example for 10 seconds once every 2 minutes for 24 hours. Blood pressure and heart rate can be routinely evaluated at baseline, for example, at 30 min, 1 h, 2 h, 4 h, 6 h, 8 h, 12 h, 16h and 20 h, etc. post-administration. Before treatment, baseline values can be taken for each animal, for example, time-point values of the last 20 minutes before the start of treatment on Day 1 are averaged and taken as the baseline value for each animal. The other time-point values can be obtained by averaging data prior and post each time-point (e.g., 10 minutes prior and 10 minutes post each time-point). A BP cut off value (e.g., 250 mmHg) and a HR cut off value (e.g., 800 bpm) can be incorporated to remove non-physiologic data points, which may be included in the raw data.

At the same time as cardiovascular recording is occurring, the motility of the animals in the metabolism cages can also be determined using the telemetry equipment. Recording can be continuous over any number of days of compound dosing.

Renal Function Monitoring

Renal function monitoring can be conducted simultaneously with cardiovascular testing. Urine is collected at scheduled time-points, for example, at 0-8 and 8-24 hour time-points. Prior to telemetry data collection, receivers equipped with special mounting strips are hung upside down on metabolism racks (Lab Products, Maywood, N.J.). Receivers that can be used include a DSI receiver. Metabolism cages are then suspended from the receivers and assembled to provide for food, water, and urine collection. Plastic tubing (e.g., 3 cm of TYGON (R-3603)) can be packed with glass wool and placed on the underside of the metabolism cage to direct the urine into appropriate collection tubes (e.g., 50 ml polypropylene conical tubes, B/D, Franklin Lakes, N.J.). Animals are weighed and placed into the metabolism cages for a baseline collection period (e.g., 30 minutes). After baseline collection, each animal is removed, dosed and returned. Pre-weighed collection tubes are placed under each metabolism cage. After a pre-determined time, e.g., 8 hours, the collection tubes are removed, weighed, and replaced with another set of pre-weighed tubes for the next time-point, e.g., 8-24 hours. After the second time-point, collection tubes are again removed and weighed. Following the second urine collection, animals are anesthetized and blood is collected.

Pulmonary Monitoring

For animals in the pulmonary/CNS arm, pulmonary function (respiratory frequency and tidal volume) can be assessed by various means known in the art including, but not limited to the BUXCO Unrestrained Whole Body Plethysmograph technique. Rats are placed in a pulmonary function recording chamber, and a baseline-recording period (e.g., 10 minutes) is collected. Following the baseline-recording period, the animals are removed from the chambers, dosed and returned for a period of data collection (e.g., 60-minutes). At the conclusion of the experiment, the animals are removed from the chambers, and tested for CNS functions.

CNS Monitoring

Additional CNS monitoring can be performed before or after pulmonary function assessment in that arm of the study. Animals can be returned to their home cages and at a specific time-point post-dosing (e.g., 65 minutes), the animals are placed in observation cylinders, e.g., Plexiglas observation cylinders. Compound treated animals are systematically compared to vehicle treated animals for behavioral, neurologic and autonomic changes according to methods known in the art, for example, FOB, Irwin profiling, i.e., a method described by Irwin (Irwin, S., 1968, Psychopharmacologia 13:222-257), or a modification thereof. Modifications of the Irwin method can include but are not limited to: performing in animals other than mice, using a different scoring system (e.g. 0, ±1, 2, 3 representing none, slight, moderate or marked effects), and not assessing all of the observations listed by Irwin. Results from this procedure can give insight into the overall profile of activity (i.e., sedation, muscle relaxation, analgesia, etc.) of a compound, in addition to its main effect and may demonstrate a need for additional testing to further elucidate such activities. Body temperatures can also be recorded both prior to dosing (baseline) and during modified Irwin testing post-dosing using a tele-thermometer (e.g., Yellow Springs Instruments Telethermometer) and rectal probe or equivalent. Alternatively, continuous body temperature may also be recorded using an alternate telemetry implant or daily temperatures may be recorded using, for example, a Bio Medic Data Systems IPTT-100 implantable programmable ID/temperature transponder (Bio Medic Data Systems, Seaford, Del.). Following modified Irwin testing, animals can be anesthetized and blood can be collected.

Blood and Tissue Collection

At the conclusion of the pulmonary function/CNS evaluation arm (single dose) and cardiovascular/renal function assessment arm (5 or more doses), blood can be collected from anesthetized animals for CBC and plasma levels of test compound. Blood can also be collected for serum clinical chemistry determinations in the cardiovascular/renal function arm. Brain samples can be taken to evaluate compound levels and determine if the compound crosses the blood-brain barrier. Other tissues (e.g., spleen, kidneys, adrenals, liver and heart/lung cluster and brain) can also be collected for histological assessment during the course of a necropsy. In addition, specific (e.g., liver, spleen and brain) can be weighed for determination of organ to body weight and organ to brain weight ratio determinations. Aside from standard histological examination, specific organs (e.g., liver, lung, bone) can be assessed using various imaging procedures (e.g., magic angle spinning NMR, PET scanning, MRI, or bone scanning using histomorphometric analysis) which in combination with metabonomics, genomics and proteomics offer further insight into likely mechanisms of toxicity. A characteristic pattern in a tissue image/scan might provide a signature of toxicity to the particular organ or region of the organ.

Data Analysis

An analysis of variance is performed with the factors: treatment, animal (according to treatment), time, and treatment time interaction; time incorporates all measurements on the given days. The adjusted mean values post-treatment are compared with baseline. If there is a significant treatment time interaction, tests are performed for comparing the three treatment groups for each time-point. If such a test is significant, the adjusted mean values of the treated group are compared with the concurrent vehicle group for this time-point.

Activity is evaluated as averaged day and night values, for example, the values of the first eleven hours after start of treatment are averaged to give a value for “day”; the values of the second eleven hours are averaged to give a value for “night”. These day and night values as well as the difference “night-day” and the percent difference from day are summarized. Statistical tests known in the art can be performed for comparing difference and percent difference in the three treatment groups (e.g., Kruskal-Wallis tests).

Renal function is routinely evaluated in the cardiovascular/renal function arm. Urine volume can be calculated gravimetrically at each time-point. Urine flow is can be calculated by (urine volume/time×100/body weight (g)). Flow values are then plotted as the mean ±standard deviation for each group. Creatinine and electrolyte (Na⁺, K⁺ and Cl⁻) concentrations can be received from the serum clinical chemistries and excretion rates are calculated by (conc./ml×flow). Excretion values are then plotted as the mean ±standard deviation for each group. Statistical significance (p<0.05) can be assessed using statistical methods known in the art such as the paired Student's t test.

The respiratory signal can be measured continuously prior to and post dosing, for example, for 10 minutes prior to dosing and 65 minutes post-dosing. Respiratory frequency and tidal volume can be then be extracted from this respiratory signal, and evaluated on a breath-by-breath basis, for example, over the course of a one-minute period. These calculated breath-by-breath values for respiratory frequency and tidal volume can be averaged over specified periods for the extent of the monitoring period, for example, over 5-minute segments. The time course for these values is then plotted as the mean ±standard deviation for each group studied. An analysis of variance similar to that described above for cardiovascular parameters can also be performed.

For body temperatures, CBC values, clinical chemistry values, plasma levels and organ weight ratios, means are calculated (± standard deviation or standard error of the mean) and reported where applicable. Statistical evaluations for compound related effects are performed using statistical methods known in the art such as the Student's t test (paired) and reported as significant if p<0.05.

Included herein are exemplified embodiments, which are intended as illustrations of single aspects of the invention. Indeed, various modifications of the invention in addition to those herein will become apparent to those skilled in the art from the foregoing description and drawings. Such modifications are intended to fall within the scope of the present invention.

All publications and patent applications cited herein are incorporated by reference in their entireties.

### EXAMPLES

Standard Protocol

Compound XYZ and several reference standards were studied using the standard protocol or a special acute dosing protocol and are shown as examples below.

Male and female Sprague Dawley rats from Charles River were used in these experiments. For the “standard” protocol, a total of 48 rats were used. Twenty-four rats (12 female rats and 12 male rats) were placed in the acute pulmonary function/CNS evaluation arm of the study, and 24 rats (12 female rats and 12 male rats) were placed in the subacute cardiovascular/renal arm of the study. Rats in both arms were further divided into 4 subgroups (3 male rats and 3 female rats) which were dosed with vehicle or compound at dose 1, dose 2; or dose 3. Rats in the pulmonary function/CNS evaluation arm of the study were administered a single dose of vehicle or compound. After dosing, a battery of pharmacologic tests including BUXCO pulmonary function assessment and modified Irwin profile assessment were performed.

Rats in the cardiovascular/renal function arm of the study (with telemetry implants) were administered daily doses of vehicle or compound for 5 days. Blood pressure, heart rate and motility were continuously monitored 24 hours/day over four days via telemetry recording. Urine was collected 0-8 and 8-24 hours post-dosing on Days 1 and 4 and assessed for changes in volume output and electrolyte excretion. On Day 5, the pulmonary function/CNS evaluations were performed on these rats. At appropriate times, blood and/or tissues were collected.

Cardiovascular and Motility Monitoring

Surgery:

For the cardiovascular/renal function arm, a Data Sciences International (DSI, St Paul, Minn.) TA11PA-C40 implant was placed in the abdominal aorta of each rat.

Cardiovascular function and motility were assessed in conscious freely moving rats using DSI telemetry recording equipment. On the day of recording, the rats were placed into metabolism cages for simultaneous urine collection. Parameters monitored included blood pressure (BP), heart rate (HR) and activity. Measurements were taken at scheduled intervals (i.e., 10 sec. of data collection once every 2 min. for each rat) over 24 hours. An appropriate baseline period of at least 30 minutes was collected prior to animal dosing. For dosing, rats were briefly removed from their cage, weighed, dosed and returned.

Renal Function Monitoring

Renal function monitoring was conducted simultaneously with cardiovascular testing. Urine was collected for 0-8 and 8-24 hour time-points. Prior to telemetry data collection, DSI receivers equipped with special mounting strips were hung upside down on metabolism racks (Lab Products, Maywood, N.J.). Metabolism cages were then suspended from the DSI receivers and assembled to provide for food, water, and urine collection. A 3 cm section of TYGON (R-3603) plastic tubing was packed with glass wool and placed on the underside of the metabolism cage to direct the urine into appropriate collection tubes (e.g., 50 ml polypropylene conical tubes, B/D, Franklin Lakes, N.J.). Rats were weighed and placed into the recording metabolism cages for a baseline collection period (i.e., 30 minutes). After baseline, each rat was removed, dosed and returned. Pre-weighed collection tubes were placed under each metabolism cage. At 8 hours, the collection tubes were removed, weighed, and replaced with another set of pre-weighed tubes for the 8-24 hour time-point. At 24 hours, collection tubes were again removed and weighed. Following the 24 hour urine collection, rats were anesthetized and blood was collected.

Pulmonary Monitoring

For rats in the pulmonary/CNS arm, pulmonary function (respiratory frequency and tidal volume) was assessed incorporating the BUXCO Unrestrained Whole Body Plethysmograph technique. Rats were placed in a BUXCO chamber, and a 10-minute baseline-recording period was collected. Following the baseline-recording period, the rats were removed from the chambers, orally dosed and returned for a 60-minute period of data collection. At the conclusion of the experiment, the rats were removed from the chambers, and tested for modified Irwin profiling.

CNS Monitoring (Motility Recording and Modified Irwin Profiling)

At the same time as cardiovascular recording was occurring, the motility of the rats in the metabolism cages was also determined using the DSI telemetry equipment. Recording is continuous over four days of compound dosing.

Additional CNS monitoring was performed following pulmonary function assessment in that arm of the study. Rats were returned to their home cages and at 65 minutes post-dosing, the rats were placed in Plexiglas observation cylinders. Compound treated rats were systematically compared to vehicle treated rats for behavioral, neurologic and autonomic changes according to a modification of the method described by Irwin. Method modifications included: performing in rats rather than mice, using a different scoring system (0, ±1, 2, 3, representing none, slight, moderate or marked effects), and not assessing all of the observations listed by Irwin. The following functions were assessed: increased/decreased motor activity, ataxia, paralysis, tremor, convulsions, abnormal respiration, enhanced salivation, skin color, abnormal feces, death, righting reflex, visual orientation, grip reflex, muscle tone, rectal body temperature, and tail pinch. Body temperatures were also recorded both prior to dosing (baseline) and during modified Irwin testing post-dosing using a Yellow Springs Instruments Telethermometer and rectal probe or equivalent. Following modified Irwin testing, rats were administered anesthesia to effect and blood was collected.

Blood and Tissue Collection

At the conclusion of the pulmonary function/CNS evaluation arm (single dose) and cardiovascular/renal function assessment arm (5 doses), blood was collected from anesthetized animals for CBC and plasma levels of test compound. Blood was also collected for serum clinical chemistry determinations in the cardiovascular/renal function arm. Brain samples were taken to evaluate compound levels and determine if the compound crossed the blood-brain barrier.

Data Analysis

Blood pressure, heart rate and activity were measured for 10 seconds once every 2 minutes for 24 hours. Blood pressure and heart rate were routinely evaluated at baseline, 30 min, 1 h, 2 h, 4 h, 6 h, 8 h, 12 h, 16 h and 20 h post-administration. The last 20 minutes before the start of treatment on Day 1, time-point values were averaged and taken as the baseline value for each rat. The other time-point values were obtained by averaging data 10 minutes prior and 10 minutes post each time-point. A BP cut off value of 250 mmHg and a HR cut off value of 800 bpm was incorporated to remove non-physiologic data points, which may be included in the raw data. An analysis of variance was performed with the factors: treatment, animal (according to treatment), time, and treatment time interaction; time incorporated all measurements on the four days. The adjusted mean values post-treatment were compared with baseline. If there was a significant treatment time interaction, tests were performed for comparing the three treatment groups for each time-point. If such a test was significant, the adjusted mean values of the treated group were compared with the concurrent vehicle group for this time-point.

Activity was evaluated as averaged day and night values. The values of the first eleven hours after start of treatment were averaged to give a value for “day”; the values of the second eleven hours were averaged to give a value for “night”. These day and night values as well as the difference “night-day” and the percent difference from day were summarized. Kruskal-Wallis tests were performed for comparing difference and percent difference in the three treatment groups.

Renal function was evaluated in the cardiovascular/renal function arm. Urine volume was calculated gravimetrically at each time-point. Urine flow was then calculated by (urine volume/time times or ×100/body weight (g)). Flow values were then plotted as the mean ±standard deviation for each group. Creatinine and electrolyte (Na⁺, K⁺ and Cl⁻) concentrations were received from the serum clinical chemistries and excretion rates were calculated by (conc./ml×flow). Excretion values were then plotted as the mean ±standard deviation for each group (data not shown). Statistical significance (p<0.05) was assessed using the paired Student's t test.

The respiratory signal was measured continuously for 10 minutes prior to dosing and 65 minutes post-dosing. Respiratory frequency and tidal volume were then extracted from this respiratory signal, and evaluated on a breath-by-breath basis over the course of a one-minute period. These calculated breath-by-breath values for respiratory frequency and tidal volume were averaged over 5-minute segments for the extent of the monitoring period. The time course for these values was then plotted as the mean ±standard deviation for each group studied. An analysis of variance similar to that described above for cardiovascular parameters was also performed.

For body temperatures, CBC values, clinical chemistry values, plasma levels and organ weight ratios, means were calculated (±standard deviation or standard error of the mean) and reported where applicable. Statistical evaluations for compound related effects were performed using the Student's t test (paired) and reported as significant if p<0.05.

### Example 1

Sprague Dawley rats 36 (18 males and 18 females; three of each sex/treatment group; acute and subacute dosing) were administered saline (control), compound XYZ (100 mg/kg, p.o.) or compound XYZ (300 mg/kg, p.o.).

Results

Results for all collected parameters are shown for a sampling of individual rats in Table 1 below.

TABLE 1
PET Rat Study - Subacute Arm Compound XYZ

| | | | | | | | Pulmonary Parameters (Day 5) | | | |
|---|---|---|---|---|---|---|---|---|---|---|
| Rat ID | | Body Weight (g) | | | | | Frequency (breaths/minute) | | Tidal Volume (mL) | |
| (# & sex) | Treatment | Day 1 | Day 2 | Day 3 | Day 4 | Day 5 | Baseline | +40 minutes | Baseline | +40 minutes |
| #2 Male | Vehicle, 5 ml/kg, po, bid | 226 | 228 | 240 | 239 | 250 | 187 | 108 | 1.54 | 1.18 |
| #3 Male | XYZ, 50 mg/kg, po, bid | 209 | 212 | 216 | 227 | 238 | 175 | 85 | 1.31 | 1.49 |
| #5 Male | XYZ, 150 mg/kg, po, bid | 219 | 193 | 225 | 234 | 243 | 309 | 107 | 1.15 | 1.40 |
| #16 Female | Vehicle, 5 ml/kg, po, bid | 211 | 221 | 216 | 223 | 224 | 154 | 75 | 1.20 | 1.20 |
| #13 Female | XYZ, 50 mg/kg, po, bid | 196 | 198 | 202 | 200 | 205 | 178 | 100 | 1.49 | 1.26 |
| #17 Female | XYZ, 150 mg/kg, po, bid | 183 | 185 | 196 | 196 | 199 | 197 | 76 | 1.30 | 1.43 |

| | | Cardiovascular Parameters (Days 1-4) | | | | | | | | | |
|---|---|---|---|---|---|---|---|---|---|---|---|
| | | Blood Pressure (mmHg) | | | | | Heart Rate (bpm) | | | | |
| Rat ID | | | Day 1 | Day 2 | Day 3 | Day 4 | | Day 1 | Day 2 | Day 3 | Day 4 |
| (# & sex) | Treatment | Baseline | +1 hr. | +1 hr. | +1 hr. | +1 hr. | Baseline | +1 hr. | +1 hr. | +1 hr. | +1 hr. |
| #2 Male | Vehicle, 5 ml/kg, po, bid | 99 | 104 | 105 | 108 | 98 | 442 | 379 | 441 | 411 | 387 |
| #3 Male | XYZ, 50 mg/kg, po, bid | 94 | 103 | 90 | 103 | 93 | 346 | 350 | 391 | 402 | 383 |
| #5 Male | XYZ, 150 mg/kg, po, bid | 100 | 98 | 119 | 106 | 96 | 380 | 382 | 468 | 464 | 398 |
| #16 Female | Vehicle, 5 ml/kg, po, bid | 101 | 107 | 106 | 94 | 111 | 362 | 385 | 383 | 407 | 493 |
| #13 Female | XYZ, 50 mg/kg, po, bid | 104 | 103 | 105 | 98 | 98 | 439 | 242 | 477 | 406 | 448 |
| #17 Female | XYZ, 150 mg/kg, po, bid | 101 | 106 | 112 | 115 | 108 | 399 | 357 | 405 | 460 | 477 |

| | | CNS - Activity Parameter (Days 1-4) | | | | | | | | |
|---|---|---|---|---|---|---|---|---|---|---|
| | | (counts) | | | | | | | | |
| Rat ID | | | Day 1 | Day 1 | Day 2 | Day 2 | Day 3 | Day 31 | Day 4 | Day 4 |
| (# & sex) | Treatment | Baseline | +1 hr. | +10 hr. | +1 hr. | +10 hr. | +1 hr. | +10 hr. | +1 hr. | +10 hr. |
| #2 Male | Vehicle, 5 ml/kg, po, bid | 0 | 0 | 0 | 0 | 0 | 0 | 0 | 0 | 0 |
| #3 Male | XYZ, 50 mg/kg, po, bid | 0 | 0 | 0 | 0 | 7 | 0.5 | 0 | 0 | 2.5 |
| #5 Male | XYZ, 150 mg/kg, po, bid | 0 | 0 | 0 | 0.5 | 0 | 0 | 0 | 0 | 0 |
| #16 Female | Vehicle, 5 ml/kg, po, bid | 0 | 1 | 0 | 0 | 0 | 0 | 3.5 | 1 | 0.5 |
| #13 Female | XYZ, 50 mg/kg, po, bid | 0 | 0 | 0 | 0 | 1 | 0 | 0 | 0 | 0 |
| #17 Female | XYZ, 150 mg/kg, po, bid | 1.5 | 0 | 0 | 0 | 1.5 | 7.5 | 0 | 3.5 | 0 |

CNS Parameters Cont. (Day 5)

| Rat ID | | Modified Irwin Profile | Body Temperature |
|---|---|---|---|
| (# & sex) | Treatment | (observations) | (° F.) |
| #2 Male | Vehicle, 5 ml/kg, po, bid | No overt effects observed at +65 min. | 99.85 |
| #3 Male | XYZ, 50 mg/kg, po, bid | No overt effects observed at +65 min. | 101.00 |
| #5 Male | XYZ, 150 mg/kg, po, bid | No overt effects observed at +65 min. | 99.60 |
| #16 Female | Vehicle, 5 ml/kg, po, bid | No overt effects observed at +65 min. | 100.65 |
| #13 Female | XYZ, 50 mg/kg, po, bid | No overt effects observed at +65 min. | 100.40 |
| #17 Female | XYZ, 150 mg/kg, po, bid | No overt effects observed at +65 min. | 100.25 |

| | | Renal Parameters | | | | | | | | | |
|---|---|---|---|---|---|---|---|---|---|---|---|
| | | Days 1-2 (0-8 hours) | | | | | Days 1-2 (8-24 hours) | | | | |
| | | | Creatinine | Na⁺ | K⁺ | Cl⁻ | | Creatinine | Na⁺ | K⁺ | Cl⁻ |
| Rat ID | | Urine Vol. | (μmole/ | (μmole/ | (μmole/ | (μmole/ | Urine Vol. | (μmole/ | (μmole/ | (μmole/ | (μmole/ |
| (# & sex) | Treatment | (ml) | ml) | ml) | ml) | ml) | (ml) | ml) | ml) | ml) | ml) |
| #2 Male | Vehicle, 5 ml/kg, po, bid | 6.6 | 16.1 | 95 | 73 | 111 | 9.4 | 36.2 | 92 | 184.2 | 144 |
| #3 Male | XYZ, 50 mg/kg, po, bid | 6.3 | 24.6 | 144 | 106.6 | 154 | 9.2 | 40.6 | 104 | 188.2 | 156 |
| #5 Male | XYZ, 150 mg/kg, po, bid | 5.2 | 31.8 | 100 | 124.4 | 128 | 7.2 | 51 | 202 | 198.6 | 208 |
| #16 Female | Vehicle, 5 ml/kg, po, bid | 3.6 | 38 | 94 | 127.8 | 134 | 8.7 | 55.6 | 164 | 248.4 | 236 |
| #13 Female | XYZ, 50 mg/kg, po, bid | 7.3 | 19.6 | 83 | 86.2 | 100 | 4.7 | 84.4 | 104 | 305.2 | 212 |
| #17 Female | XYZ, 150 mg/kg, po, bid | 3.5 | 17.8 | 94 | 67.6 | 108 | 5.6 | 61.2 | 176 | 220 | 216 |
| | | Renal Parameters | | | | | | | | | |
| | | Days 4-5 (0-8 hours) | | | | | Days 4-5 (8-24 hours) | | | | |
| | | | Creatinine | Na⁺ | K⁺ | Cl⁻ | | Creatinine | Na⁺ | K⁺ | Cl⁻ |
| Rat ID | | Urine Vol. | (μmole/ | (μmole/ | (μmole/ | (μmole/ | Urine Vol. | (μmole/ | (μmole/ | (μmole/ | (μmole/ |
| (# & sex) | Treatment | (ml) | ml) | ml) | ml) | ml) | (ml) | ml) | ml) | ml) | ml) |
| #2 Male | Vehicle, 5 ml/kg, po, bid | 4.8 | 45.2 | 168 | 178.6 | 190 | 8.9 | 58.8 | 68 | 291.2 | 188 |
| #3 Male | XYZ, 50 mg/kg, po, bid | 6.0 | 35.6 | 188 | 129 | 192 | 10.2 | 58.4 | 120 | 303.6 | 236 |
| #5 Male | XYZ, 150 mg/kg, po, bid | 4.7 | 40.6 | 198 | 168.4 | 202 | 7.3 | 61.6 | 180 | 315.6 | 264 |
| #16 Female | Vehicle, 5 ml/kg, po, bid | 4.8 | 41.2 | 144 | 208 | 228 | 5.0 | 67.6 | 116 | 329.6 | 228 |
| #13 Female | XYZ, 50 mg/kg, po, bid | 3.4 | 54.8 | 76 | 239.6 | 188 | 6.2 | 76 | 124 | 362.8 | 256 |
| #17 Female | XYZ, 150 mg/kg, po, bid | 3.9 | 46 | 124 | 206 | 200 | 5.5 | 66.4 | 132 | 335.6 | 252 |

| | | Blood Parameters (Days 5) | | | | | | | | | | | | | |
|---|---|---|---|---|---|---|---|---|---|---|---|---|---|---|---|
| | | CBC | | | | | | | | | | | | | |
| | | Total | Differential Counts | | | | | | | | | | | | |
| Rat ID | | WBC | % | % | % | Clinical Chemistry Values (serum) | | | | | | | | | |
| (# & sex) | Treatment | (× 10³/μL) | Lymphs | Neuts | Eos | Creat. | Na⁺ | K⁺ | Cl⁻ | Glu. | BUN | ALP | ALT | AST | TP |
| #2 Male | Vehicle, 5 ml/kg, po, bid | 9.6 | 61 | 38 | 1 | 0.4 | 148 | 4.8 | 107 | 139 | 10.9 | 268 | 55 | 204 | 5.3 |
| #3 Male | XYZ, 50 mg/kg, po, bid | 3.6 | 79 | 21 | 0 | 0.4 | 147 | 5.2 | 107 | 171 | 24.8 | 283 | 81 | 372 | 5.3 |
| #5 Male | XYZ, 150 mg/kg, po, bid | 13.3 | 67 | 33 | 0 | 0.4 | 148 | 4.1 | 108 | 182 | 19.9 | 243 | 50 | 181 | 5.0 |
| #16 Female | Vehicle, 5 ml/kg, po, bid | 4.5 | 80 | 20 | 0 | 0.4 | 158 | 4.6 | 114 | 145 | 14.2 | 218 | 54 | 208 | 5.7 |
| #13 Female | XYZ, 50 mg/kg, po, bid | 12.5 | 63 | 35 | 2 | * | * | * | * | 142 | 17.2 | 132 | 52 | 310 | 5.8 |
| #17 Female | XYZ, 150 mg/kg, po, bid | 11.1 | 84 | 16 | 0 | 0.4 | 158 | 5.2 | 115 | 120 | 10.9 | 196 | 61 | 310 | 5.4 |

* = missing value

| | | Blood Param. Cont. | Organ Weights (Day 5) | | | Organ Weight Ratios (Day 5) | | | |
|---|---|---|---|---|---|---|---|---|---|
| Rat ID | | Plasma Levels (Day 5) | Liver | Spleen | Brain | Liver to | Liver to | Spleen to | Spleen to |
| (# & sex) | Treatment | (ng/mL) | Weight (g) | Weight (g) | Weight (g) | Body Wt. | Brain Wt. | Body Wt. | Brain Wt. |
| #2 Male | Vehicle, 5 ml/kg, po, bid | BDL | 4.4922 | 0.5264 | 1.8638 | 0.0180 | 2.4102 | 0.0021 | 0.2824 |
| #3 Male | XYZ, 50 mg/kg, po, bid | 163.5 | 9.0778 | 0.5803 | 1.6464 | 0.0381 | 5.5137 | 0.0024 | 0.3525 |
| #5 Male | XYZ, 150 mg/kg, po, bid | 372.7 | 9.5109 | 0.6939 | 1.7843 | 0.0391 | 5.3303 | 0.0029 | 0.3889 |
| #16 Female | Vehicle, 5 ml/kg, po, bid | BDL | 9.2405 | 0.5646 | 1.8150 | 0.0412 | 5.0912 | 0.0025 | 0.3111 |
| #13 Female | XYZ, 50 mg/kg, po, bid | 749.5 | 9.1311 | 0.6288 | 1.8098 | 0.0445 | 5.0454 | 0.0031 | 0.3474 |
| #17 Female | XYZ, 150 mg/kg, po, bid | 5,067.4 | 8.9614 | 0.4822 | 1.9887 | 0.0451 | 4.5062 | 0.0024 | 0.2425 |

BDL = below detectable limits

The reference standards studied using the standard protocol include: d-amphetamine, an adrenergic agonist with stimulant properties; diazepam, a benzodiazepine with sedative and muscle relaxing properties; prednisolone, an adrenocortical steroid with anti-inflammatory properties; furosemide, a high ceiling or loop diuretic.

### Example 2

Sprague Dawley rats 36 (18 males and 18 females; three of each sex/treatment group; acute and subacute dosing) were administered saline (control) (5 ml/kg, p.o., -▪-), d-amphetamine (2 mg/kg, p.o., -●-) or d-amphetamine (8 mg/kg, p.o, -▴-).

Results

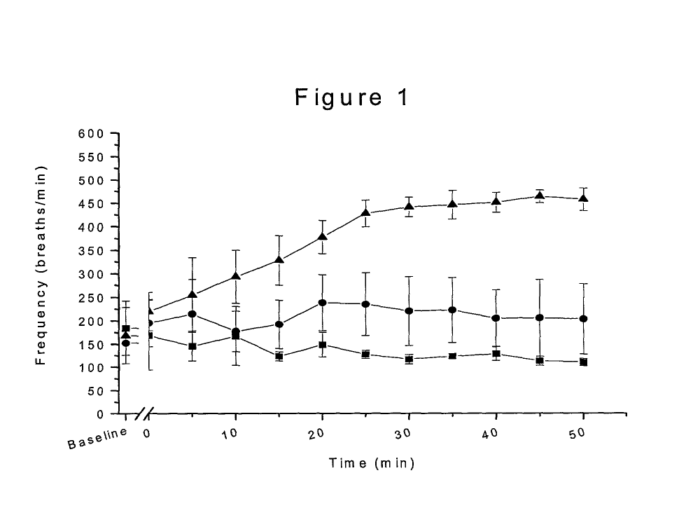

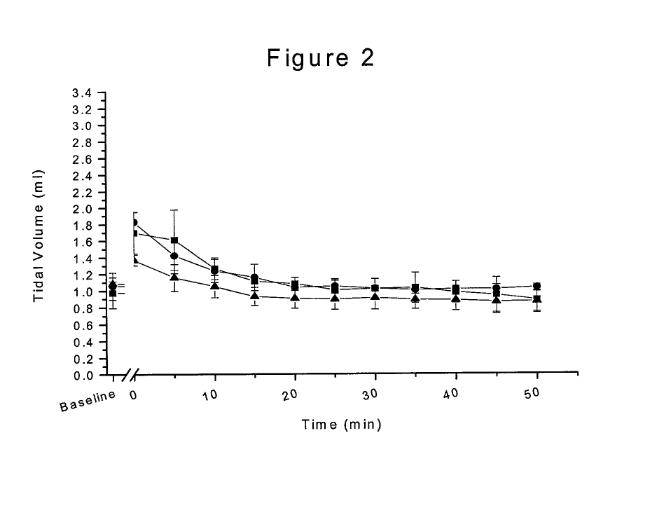

Rats administered d-amphetamine exhibited increased motility, sniffing, rearing, head weaving and vocalization (male and female; acute and subacute) (data not shown). Motility was increased during the day as compared to the night (see Table 2 showing data for female rats). Observed cardiovascular effects included slightly increased BP and HR for males at 8 mg/kg doses (data not shown). As shown in FIG. 1, respiratory frequency increased in a dose dependent manner (male and female; following acute and subacute dosing; male acute data only shown). As shown in FIG. 2, no relevant changes in mean tidal volume were demonstrated for the same treatment groups (male acute data only shown).

TABLE 2

| | | 2 mg/kg | 8 mg/kg |
|---|---|---|---|
| day | period | % Change from Control | % Change from Control |
| 1 | day | +391* | +194* |
| | night | +8 | 0 |
| 2 | day | +405* | +175* |
| | night | +5 | −22 |
| 3 | day | +390* | +182* |
| | night | −31 | −29 |
| 4 | day | +320* | +255* |
| | night | −47 | −55 |

*significant p <0.05

### Example 3

Sprague Dawley rats 36 (18 males and 18 females; three of each sex/treatment group; acute and subacute dosing) were administered 0.5% methocel (control) (5 ml/kg, p.o.), diazepam at 20 mg/kg, p.o. or 80 mg/kg, p.o. doses. (Data not shown).

Results

In the acute arm of the study, modified Irwin profiling showed dose dependent effects on motility, coordination, muscle tone and body temperature. In the subacute arm of the study, modified Irwin profiling showed dose dependent effects on motility, coordination, muscle tone and body temperature. No relevant effects were observed in the CV or the pulmonary system.

### Example 4

Sprague Dawley rats 36 (18 males and 18 females; three of each sex/treatment group; acute and subacute dosing) were administered CMC/Tween 80 (1%/0.2%) (control) (5ml/kg, p.o), prednisolone at 300 mg/kg or prednisolone at 1000 mg/kg, p.o.

Results

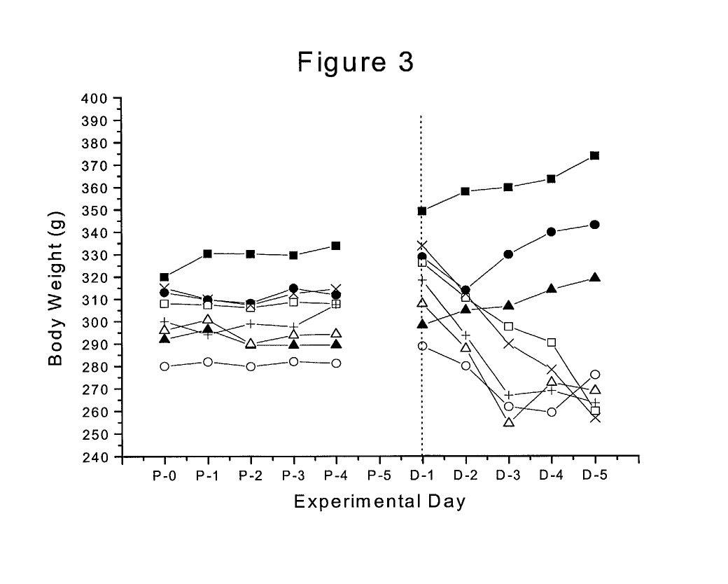

No relevant effects were observed in the CNS, CV, or pulmonary systems. As shown in FIG. 3, a decrease in body weight was observed in both males and females (males only shown). In addition, effects on the liver were also demonstrated including: increased liver to body and liver to brain weight ratio, increases in liver enzymes (aspartate aminotransferase (AST) and alanine aminotransferase (ALT)) and abnormal liver morphology upon necropsy (data not shown). A dose dependent neutrophilia, an increase in the number of neutrophils and a corresponding decrease in lymphocytes, was also observed in males (data not shown).

### Example 5

Sprague Dawley rats 36 (18 males and 18 females; three of each sex/treatment group; acute dosing) were administered 0.5% methocel (control) (5 ml/kg, p.o.), furosemide at 10 mg/kg, p.o. or 30 mg/kg, p.o.

Results

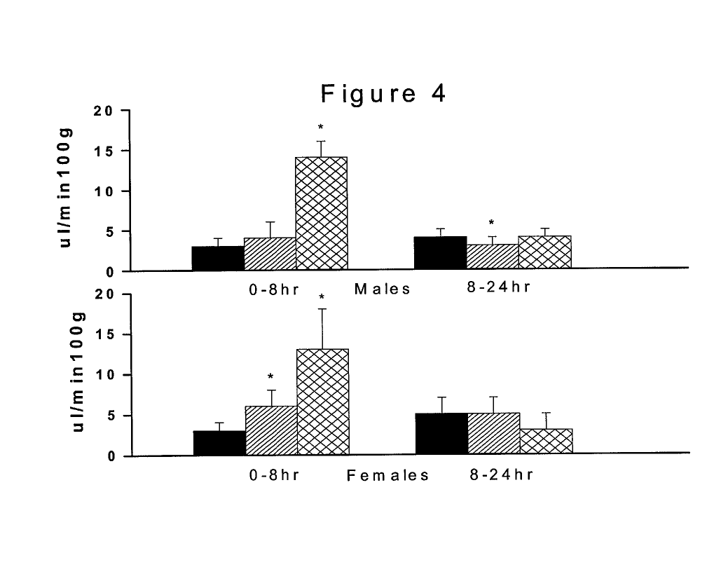

As shown in FIG. 4, furosemide dose dependently increased urine output in both males and females at the doses tested. In addition, a dose dependent increase in Na⁺ excretion (natriuresis) and Cl⁻ excretion was also observed in the 0-8 hour urine collection period (data not shown).

Another reference compound, nifedipine, a calcium channel blocker with hypotensive effects was studied in a separate acute dosing protocol.

### Example 6

Wistar rats 20 (5/treatment group; acute dosing) were administered 1% natrosol 250 (control)(5 ml/kg, p.o.), or nifedipine at 1, 3 or 10 mg/kg, p.o.

Results

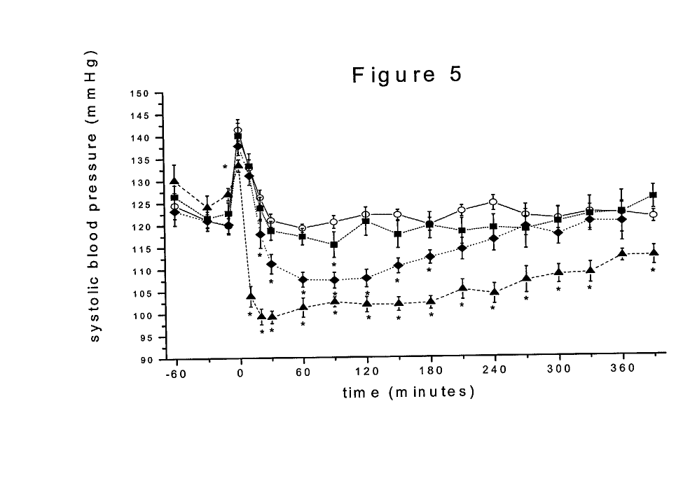

As depicted in FIG. 5, nifedipine dose dependently decreased arterial blood pressure. A concomitant dose dependent increase in heart rate was also observed, which was thought to be a reflex response to the fall in blood pressure (data not shown). No effects of nifedipine were noted on respiration, motility or body temperature. (Schierok, H et. al. 2000, J. Pharm. Tox. Methods 43:211-217.)

What is claimed is:

1. A data collection system for performing simultaneous cardiovascular and renal pharmacological evaluation tests in a single animal comprising:
(a) implanting a transmitting device into an animal;
(b) placing the animal into a recording metabolism cage;
(c) collecting urine from the animal for determining baseline renal function data;
(d) transmitting baseline cardiovascular function data by means of the transmitting device;
(e) receiving, inputting and storing the baseline data onto a storage medium;
(f) administering a substance of interest to the animal;
(g) transmitting cardiovascular function data by means of the transmitting device;
(h) collecting urine for determining renal function data following administration of the substance;
(i) receiving, inputting and storing the cardiovascular and renal function data onto a storage medium; and
(j) processing the cardiovascular and renal function data.

2. A data collection system for performing simultaneous cardiovascular, motility and renal pharmacological evaluation tests in a single animal comprising:
(a) implanting a transmitting device into an animal;
(b) placing the animal into a recording metabolism cage;
(c) collecting urine from the animal for determining baseline renal function data;
(d) transmitting baseline cardiovascular and motility function data by means of the transmitting device;
(e) receiving, inputting and storing the baseline data onto a storage medium;
(f) administering a substance of interest to the animal;
(g) transmitting cardiovascular and motility function data by means of the transmitting device;
(h) collecting urine for determining renal function data following administration of the substance;
(i) receiving, inputting and storing the cardiovascular, motility and renal function data onto a storage medium; and
(j) processing the cardiovascular, motility and renal function data.

3. The method according to claim 1 wherein steps (f), (g), (h), (i), and (j) are repeated.

4. The method according to claim 2 wherein steps (f), (g), (h), (i), and (j) are repeated.

5. A data collection system for performing simultaneous cardiovascular, renal, central nervous and pulmonary pharmacological evaluation tests in a single animal comprising:
(a) implanting a transmitting device into an animal;
(b) placing the animal into a recording metabolism cage;
(c) collecting urine from the animal for determining baseline renal function data;
(d) transmitting baseline cardiovascular function data by means of the transmitting device;
(e) receiving, inputting and storing the baseline data onto a storage medium;
(f) administering a substance of interest to the animal;
(g) transmitting cardiovascular function data by means of the transmitting device;
(h) collecting urine for determining renal function data following administration of the substance;
(i) receiving, inputting and storing the cardiovascular and renal function data onto a storage medium;
(j) processing the cardiovascular and renal function data;
(k) placing the animal into a pulmonary function recording chamber;
(l) recording baseline pulmonary function data;
(m)administering the substance of interest to the animal;
(n) recording pulmonary function data following administration of the substance;
(o) placing the animal into an observation chamber; and
(p) monitoring central nervous system function.

6. A data collection system for performing simultaneous cardiovascular, motility, renal, central nervous and pulmonary pharmacological evaluation tests in a single animal comprising:
(a) implanting a transmitting device into an animal;
(b) placing the animal into a recording metabolism cage;
(c) collecting urine from the animal for determining baseline renal function data;
(d) transmitting baseline cardiovascular and motility function data by means of the transmitting device;
(e) receiving, inputting and storing the baseline data onto a storage medium;
(f) administering a substance of interest to the animal;
(g) transmitting cardiovascular and motility function data by means of the transmitting device;
(h) collecting urine for determining renal function data following administration of the substance;
(i) receiving, inputting and storing the cardiovascular, motility and renal function data onto a storage medium;
(j) processing the cardiovascular, motility and renal function data
(k) placing the animal into a pulmonary function recording chamber;
(l) recording baseline pulmonary function data;
(m)administering the substance of interest to the animal;
(n) recording pulmonary function data following administration of the substance;
(o) placing the animal into an observation chamber; and
(p) monitoring central nervous system function.
